(19) United States
(12) Patent Application Publication
(10) Pub. No.: US 2009/0220377 A1
(43) Pub. Date: Sep. 3, 2009

(54) ENDOSCOPE WASHING AND DISINFECTING APPARATUS AND ENDOSCOPE WASHING AND DISINFECTING METHOD

(75) Inventors: Hitoshi HASEGAWA, Hamburg (DE); Shinichiro KAWACHI, Tokyo (JP); Eiri SUZUKI, Sagamihara-shi (JP); Kenichi KOBAYASHI, Tokyo (JP); Keisuke NOZAKI, Zama-shi (JP); Toshiaki NOGUCHI, Tokyo (JP); Hideto ONISHI, Tokyo (JP); Hisashi KUROSHIMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/388,814

(22) Filed: Feb. 19, 2009

(30) Foreign Application Priority Data
Feb. 27, 2008 (JP) 2008-046645
Jan. 28, 2009 (JP) 2009-017356

(51) Int. Cl. A61L 2/18

(52) U.S. Cl. 422/28; 422/292; 422/105

(57) ABSTRACT

An endoscope washing and disinfecting apparatus includes: a fluid supply unit that supplies fluid for washing and disinfecting; an electromagnetic valve provided in each of a plurality of connecting channels which are connected to a plurality of channels of an endoscope; a single flow rate meter provided between the fluid supply unit and the electromagnetic valve; and a flow rate limiting section for limiting flow rate so that the flow rate falls within a flow rate measurement range in which flow rate measurement by the flow rate meter is possible; or a flow rate padding section for padding a flow rate with a flow rate that can be detected by the flow rate meter; or a flow rate diverting section for diverting part of flow rate that flows to the flow rate meter through a bypass channel.

This application claims benefit of Japanese Application Nos. 2008-046645 filed in Japan on Feb. 27, 2008 and 2009-017356 filed in Japan on Jan. 28, 2009, the contents of which are incorporated by this reference.

### BACKGROUND OF THE INVENTION

1. Field of the Invention

The present invention relates to an endoscope washing and disinfecting apparatus and an endoscope washing and disinfecting method for washing and disinfecting a plurality of channels provided in an endoscope.

2. Description of Related Art

In recent years, endoscopes have come to be widely used in the fields of medicine or the like. After being used for an endoscopic examination, an endoscope is subjected to processing for washing and disinfecting in an endoscope washing and disinfecting apparatus to get cleaned up so that it can be reused in a clean condition.

Some endoscope washing and disinfecting apparatuses have flow control function (or flow rate control function) for checking a flow rate in each channel of an endoscope and judging the ability to wash and disinfect.

For instance, Japanese Patent Application Laid-Open Publication No. 2001-299697 discloses an endoscope washing and disinfecting apparatus that measures flow rates in gas/water supply channels and suction channels of an endoscope during washing and disinfecting operations for the channels by means of a flow rate sensor serving as a flow rate meter, and determines whether a measured flow rate is within a range of a set value so as to control fluid in the channels of the endoscope.

### SUMMARY OF THE INVENTION

An endoscope washing and disinfecting apparatus according to an embodiment of the present invention includes:

a fluid supply unit that supplies fluid for washing and disinfecting;

a plurality of connecting channels which are connected to a plurality of channels of an endoscope;

an electromagnetic valve provided in each of the plurality of connecting channels;

a single flow rate meter, provided between the fluid supply unit and the electromagnetic valve; and

a flow rate limiting section for limiting flow rate to at least a channel in which the fluid flows at a flow rate exceeding a flow rate measurement range in which flow rate measurement by the flow rate meter is possible among the plurality of channels so that the flow rate falls within the flow rate measurement range; or a flow rate padding section for padding a flow rate measured by the flow rate meter with a flow rate that can be detected by the flow rate meter at least for a channel in which the fluid flows at a flow rate that does not reach a lower limit value of the flow rate measurement range among the plurality of channels; or a flow rate diverting section for diverting part of flow rate that flows to the flow rate meter through a bypass channel which is opened and closed in parallel with the flow rate meter at least for a channel in which the fluid flows at a flow rate exceeding the flow rate measurement range among the plurality of channels, so that flow rate falls within the flow rate measurement range of the flow rate meter.

An endoscope washing and disinfecting method according to an embodiment of the present invention for washing and disinfecting a plurality of channels of an endoscope with fluid supplied from a fluid supply unit includes:

a flow rate monitoring step of monitoring a flow rate in each of the plurality of channels by means of a single flow rate meter provided between the fluid supply unit and an electromagnetic valve provided in each of a plurality of connecting channels connected to each of the plurality of channels, wherein

the flow rate monitoring step employs:

a flow rate limiting step of limiting flow rate to at least a channel in which the fluid flows at a flow rate exceeding a flow rate measurement range in which flow rate measurement by the flow rate meter is possible among the plurality of channels so that the flow rate falls within the flow rate measurement range; or

a flow rate padding step of padding a flow rate measured by the flow rate meter with a flow rate that can be detected by the flow rate meter at least for a channel in which the fluid flows at a flow rate that does not reach a lower limit value of the flow rate measurement range among the plurality of channels; or

a flow rate diverting step of diverting part of flow rate that flows to the flow rate meter through a bypass channel which is opened and closed in parallel with the flow rate meter so that flow rate falls within the flow rate measurement range of the flow rate meter.

### BRIEF DESCRIPTION OF THE DRAWINGS

FIG. 1 shows an overall configuration of an endoscope washing and disinfecting apparatus according to a first embodiment of the present invention;

FIG. 2 schematically shows a configuration of channels of an endoscope;

FIG. 3 is a block diagram showing a configuration of a flow rate control section of FIG. 1;

FIG. 4 is a flowchart showing a representative example of a processing procedure for a washing and disinfecting process in the first embodiment;

FIG. 5 is a block diagram showing a configuration of the flow rate control section in a second embodiment of the present invention;

FIG. 6 is a timing chart for illustrating operations in the second embodiment;

FIG. 7 is a block diagram showing a configuration of the flow rate control section in a third embodiment of the present invention;

FIG. 8 illustrates operations in the third embodiment;

FIG. 9 is a block diagram showing a configuration of the flow rate control section in a first variation of the third embodiment;

FIG. 10 illustrates operations in the first variation;

FIG. 11 is a block diagram showing a configuration of the flow rate control section in a second variation of the third embodiment;

FIG. 12 is a block diagram showing a configuration of the flow rate control section in a third variation of the third embodiment;

FIG. 13 shows an overall configuration of the endoscope washing and disinfecting apparatus according to a fourth embodiment of the present invention;

FIG. 14 shows an overall configuration of the endoscope washing and disinfecting apparatus according to a fifth embodiment of the present invention;

FIG. 15 is a flowchart illustrating a portion of a processing procedure for a washing and disinfecting process in the fifth embodiment;

FIG. 16 is a flowchart illustrating a processing procedure for measuring and storing a liquid supply rate of a pump in the fifth embodiment; and

FIG. 17 illustrates operations in a washing process in the fifth embodiment.

### DETAILED DESCRIPTION OF PREFERRED EMBODIMENTS

Hereinafter, embodiments of the present invention will be described with reference to drawings.

### First Embodiment

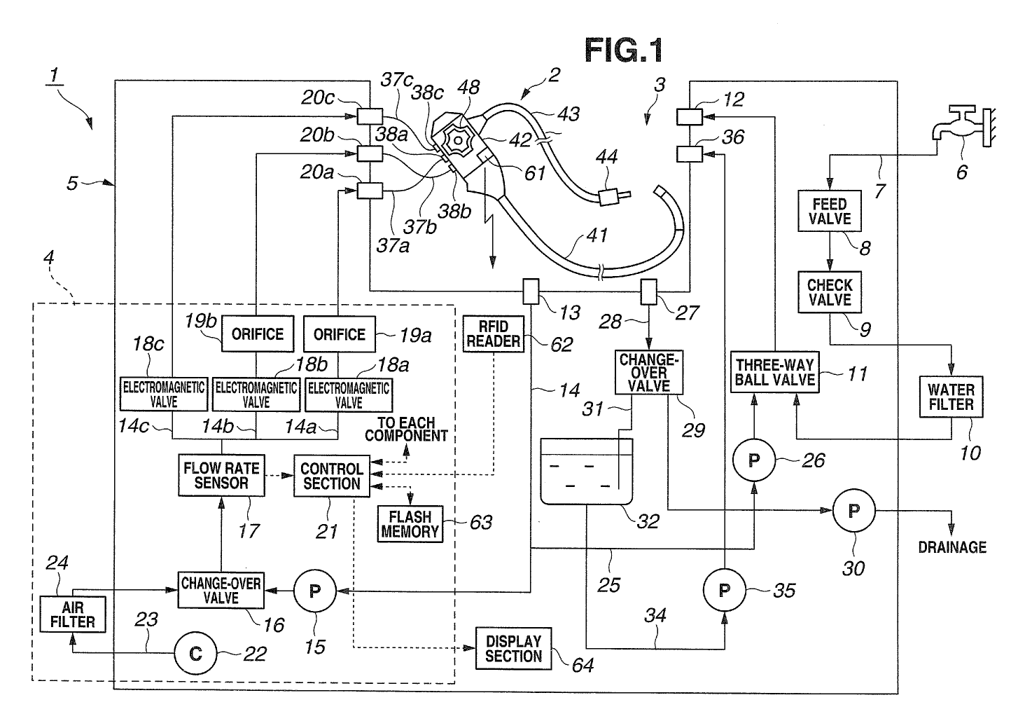

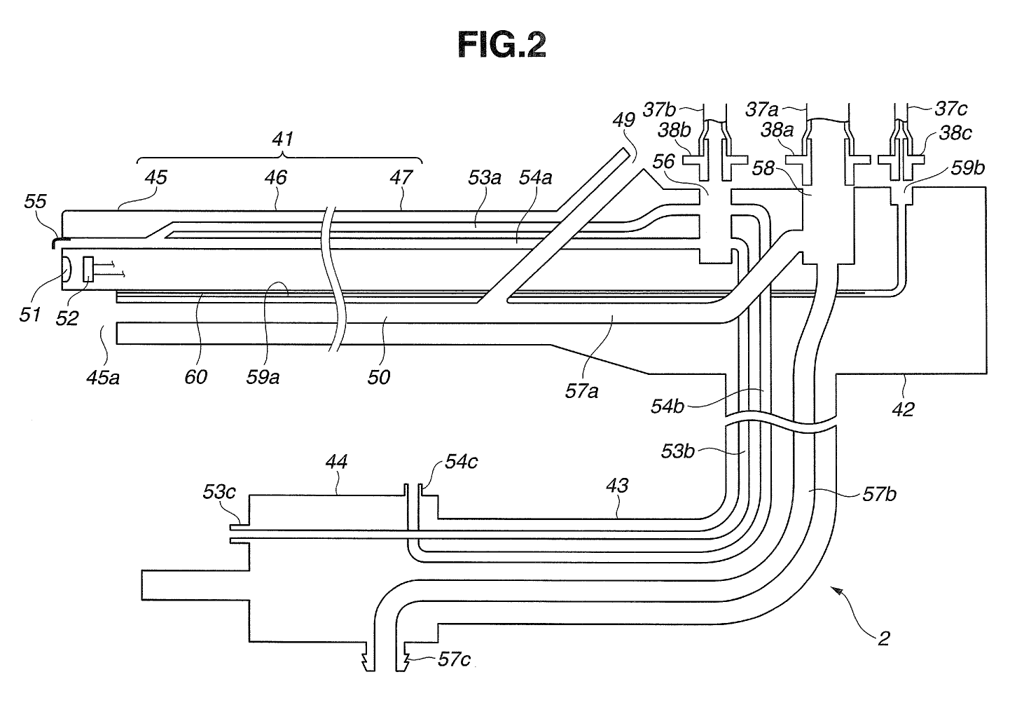

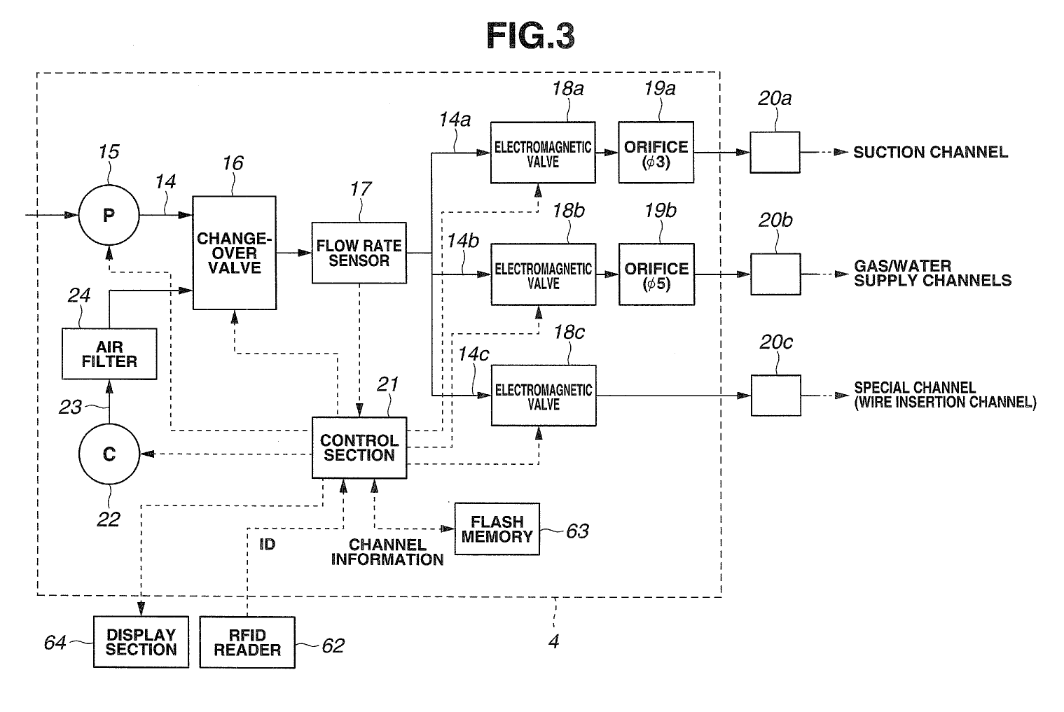

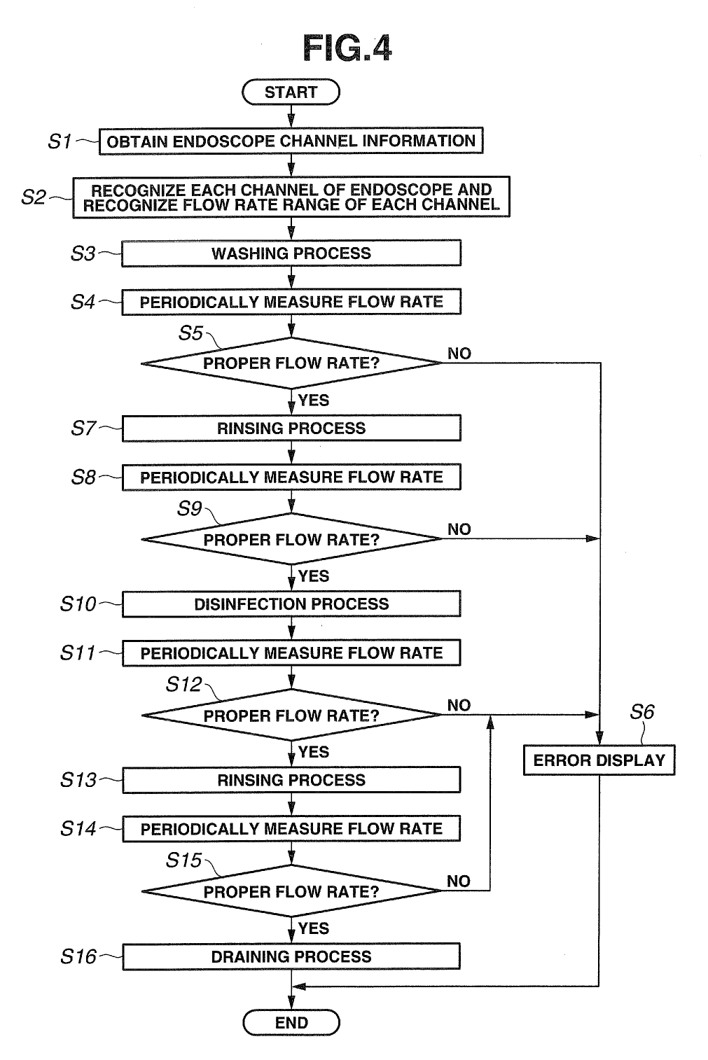

FIGS. 1 to 4 relates to a first embodiment of the present invention: FIG. 1 shows an overall configuration of an endoscope washing and disinfecting apparatus according to the first embodiment of the present invention; FIG. 2 shows a schematic configuration of channels of an endoscope; FIG. 3 shows a configuration of a flow rate control section of FIG. 1; and FIG. 4 shows a representative example of a processing procedure for washing and disinfecting process in the first embodiment.

As illustrated in FIG. 1, an endoscope washing and disinfecting apparatus 1 of the first embodiment of the invention has a washing and disinfecting bath 3 in which an endoscope 2 which should be washed and disinfected (denoted as just “washed/disinfected”) is placed and washed/disinfected with fluid, and an endoscope washing and disinfecting apparatus main body (hereinafter referred to as just “main body”) 5 which is provided around the washing and disinfecting bath 3 and which includes a flow rate control section 4 for controlling flow rate during washing/disinfecting of the channels of the endoscope 2 and other components.

To a water supply 6 such as a faucet, a first liquid supply channel 7 is connected, and water supplied from the water supply 6 for use as washing water fluid goes through a feed valve 8 and a check valve 9 which are provided midway in the first liquid supply channel 7 and filtered through a water filter 10, which is provided, for example, on a side surface of the main body 5 in a replaceable manner.

Water cleaned by this filtering is supplied as washing water via a three-way ball valve 11 to inside the washing and disinfecting bath 3 from a liquid supply port 12 which is provided, for example, on a side surface of the washing and disinfecting bath 3.

To a first drain port 13 provided, for example on a bottom surface of the washing and disinfecting bath 3, one end of a channel 14 is connected. Fluid such as washing water or disinfectant from the washing and disinfecting bath 3 that flows in the channel 14 is sent or supplied to a plurality of channels of the endoscope 2 via a pump 15 that forms a fluid supply unit.

Washing water and/or disinfectant from the washing and disinfecting bath 3 is supplied to the side of a flow rate sensor 17, which serves as a flow rate meter for measuring flow rate, via a change-over valve 16 by the pump 15 which is provided midway in the channel 14. The flow rate sensor 17 measures or detects the flow rate of fluid flowing in the channel 14. The channel 14 in which the flow rate sensor 17 is provided midway is further connected to electromagnetic valves 18a, 18b, and 18c via a plurality of branched channels 14a, 14b, and 14c.

Each channel 14i of the plurality of channels 14a to 14c to which electromagnetic valve 18i (i=a to c) is connected forms a connecting channel which is connected to one of channels of the endoscope 2 as discussed below. Then, via the channel 14i through electromagnetic valve 18i which is opened, washing water or disinfectant is supplied to a channel of the endoscope 2.

The other ends of the channels 14a and 14b, in which the electromagnetic valves 18a and 18b are inserted respectively, are further connected with a suction channel connecting mouthpiece 20a and an gas/water supply channel connecting mouthpiece 20b, which are provided on a side surface of the washing and disinfecting bath 3, via orifices (valves) 19a and 19b which are inserted midway and form flow rate limiting sections for limiting flow.

Also, the other end of the channel 14c in which the electromagnetic valve 18c is inserted is connected to a special channel connecting mouthpiece 20c provided on a side surface of the washing and disinfecting bath 3.

A flow rate measured by the flow rate sensor 17 is inputted via a signal line to a control section 21 which has functions as means for controlling the flow rate control section 4 as well as functions as means for controlling the entire endoscope washing and disinfecting apparatus 1.

In FIG. 1 and other figures, signal lines are denoted by dotted lines and channels and the like are denoted by solid lines. Also, as shown in FIG. 3, opening/closing of electromagnetic valve 18i or the like is controlled by the control section 21.

The change-over valve 16 is also connected with a channel 23 into which air is supplied from a compressor 22. When the change-over valve 16 is switched to a channel 23 on the side of the compressor 22 by the control section 21, air as fluid supplied from the compressor 22 is filtered through an air filter 24 provided midway in the channel 23 to become clean air and then flows into the channel 14 which communicates with the channel 23 (and in which the flow rate sensor 17 is inserted).

The channel 14 connected to the first drain port 13 is also connected with a channel 25 which branches on the way to the pump 15, and a pump 26 is inserted midway in the channel 25. Liquid flowing in the channel 25 is drawn by the pump 26 and brought back to the washing and disinfecting bath 3 from the liquid supply port 12 via the three-way ball valve 11. The pump 26 circulates washing/disinfecting fluid to enable continuous washing/disinfecting.

Also, to a second drain port 27 which is provided, for example on the bottom surface of the washing and disinfecting bath 3, one end of a channel 28 is connected. Washing water or disinfectant from the washing and disinfecting bath 3 that flows in the channel 28 is coupled to a drain pump 30 via a change-over valve 29 provided on the way and also coupled to a disinfectant tank 32 via a branched channel 31.

When washing water in the washing and disinfecting bath 3 has become unclean after a washing process and is to be drained, it is drained from a drain port via the drain pump 30.

Also, disinfectant from the washing and disinfecting bath 3 is once stored in the disinfectant tank 32 via the change-over valve 29, and drawn by a disinfectant pump 35 which is inserted midway in the channel 34 which is connected, for example, on the bottom of the disinfectant tank 32, to be brought back into the washing and disinfecting bath 3 from a second liquid supply port 36.

The suction channel connecting mouthpiece 20a, gas/water supply channel connecting mouthpiece 20b, and special channel connecting mouthpiece 20c are connected to connecting sections (e.g., cylinders) of a suction channel, a gas supply channel and a water supply channel (sometimes referred to as “gas/water supply channels” for short), and a special channel of the endoscope 2, respectively, via connecting tubes 37a, 37b, and 37c. Connecting mouthpieces 38a, 38b, and 38c at the ends of the connecting tubes 37a, 37b and 37c are connected to the connecting sections of the suction channel, gas/water supply channel, and special channel of the endoscope 2, respectively.

The endoscope 2 has an insertion portion 41 which has an elongated shape, an operation portion 42 which is provided at a rear end of the insertion portion 41, and a universal cable 43 which extends from a side surface of the operation portion 42. A connector 44 at an end of the universal cable 43 is connected to a light source device not shown and a video processor serving as a signal processing device.

The insertion portion 41 has a distal end portion 45 provided at an end of the insertion portion 41, a bending portion 46 which is bendable, and a flexible portion 47 which is elongated and has flexibility (see FIG. 2 for reference numerals). A user, such as an operator, can bend the bending portion 46 in a desired direction by manipulating a bending knob 48 provided on operation portion 42.

Near a front end of the operation portion 42, a treatment instrument insertion port 49 (see FIG. 2) for inserting a treatment instrument is provided. The treatment instrument insertion port 49 internally communicates with a treatment instrument channel 50 (see FIG. 2) which is provided inside the insertion portion 41.

The flow rate control section 4 has flash memory 63, for example, that has stored therein control program information for a CPU constituting the control section 21 to perform control operations, for example, and/or information in channels of various endoscopes 2.

FIG. 2 shows a general configuration of a channel system relating to washing/disinfecting in the endoscope 2.

At a distal end portion 45 of the insertion portion 41, an observation window is provided adjacent to an illumination window not shown, and an object lens 51 is attached on the observation window. At an image forming position of the object lens 51, a charge coupled device (abbreviated as CCD) 52 is arranged. The CCD 52 is connected to a signal line, which signal line is connected to an electric contact not shown of a connector 44 via the insertion portion 41, operation portion 42, and universal cable 43.

Inside the insertion portion 41, a gas supply channel 53a and a water supply channel 54a are provided in a longitudinal direction of the insertion portion 41, and the channels 53a and 54a join into one channel near the distal end portion and open at a distal-end nozzle 55 on a distal-end surface. The distal-end nozzle 55 is provided such that the nozzle 55 faces an outer surface of the object lens 51.

The rear ends of the gas supply channel 53a and the water supply channel 54a open on a gas/water supply channel cylinder 56 of the operation portion 42.

The gas supply channel 53a and the water supply channel 54a communicate, in the gas/water supply channel cylinder 56, with a gas supply channel 53b and a water supply channel 54b which are inserted through the universal cable 43. The gas supply channel 53b and water supply channel 54b which are inserted through the universal cable 43 open at a gas supply mouthpiece 53c and a water supply mouthpiece 54c, respectively, of the connector 44.

The channel of a treatment instrument channel 50 provided in the insertion portion 41 branches near the front end of the operation portion 42 to communicate with the treatment instrument insertion port 49 and further is extended into the rear side of the operation portion 42 to communicate with a suction channel 57a.

The suction channel 57a opens on a suction channel cylinder 58 which is provided in the operation portion 42. The suction channel 57a then communicates, in the suction channel cylinder 58, with a suction channel 57b which is inserted through the universal cable 43.

The suction channel 57b inserted through the universal cable 43 opens at a suction mouthpiece 57c on the connector 44.

At an opening 45a provided at the distal end portion 45 of the insertion portion 41, a treatment instrument raising stand not shown (hereinafter referred to as just a “raising stand”) is arranged. To the raising stand, a distal end of a raising operation wire 60 is coupled that is inserted through a raising wire insertion channel (hereinafter referred to just as a wire insertion channel) 59a which is provided inside the insertion portion 41.

The rear end of the raising operation wire 60 inserted through the wire insertion channel 59a is coupled to a raising operation knob not shown on the operation portion 42. Also, the wire insertion channel 59a opens in a wire insertion channel cylinder (or mouthpiece) 59b on the operation portion 42.

The operator can protrude forward a distal end of a treatment instrument inserted from the treatment instrument insertion port 49 from the opening 45a, which opens at a distal end, through the treatment instrument channel 50. In this situation, when the operator manipulates the raising operation knob to pull the raising operation wire 60, for example, the raising stand lifts up and the direction in which the distal end of the treatment instrument protrudes can be changed.

The wire insertion channel 59a through which the raising operation wire 60 is inserted is formed of a channel of a smaller inner diameter than that of the gas supply channel 53a or the water supply channel 54a. In addition, because the raising operation wire 60 is inserted inside the wire insertion channel 59a, the channel diameter of a substantial hollow portion in the wire insertion channel 59a is very small.

In general, the treatment instrument channel 50 is formed of a channel having a quite larger inner diameter than that of the gas supply channel 53a or the water supply channel 54a.

Thus, the endoscope 2 includes multiple types of channels with varying inner diameters.

For example, as mentioned above, the connecting mouthpieces 38a, 38b, and 38c of the connecting tubes 37a, 37b and 37c are connected to the suction channel cylinder 58, gas/water supply channel cylinder 56, and wire insertion channel cylinder 59b, respectively.

The endoscope 2 also has, on the operation portion 42 or the like, an RFID tag 61 as identification information generation means in which identification information (abbreviated as ID) specific to the endoscope 2 is written.

An ID stored in memory inside the RFID tag 61 is read by an RFID reader 62, which is provided inside the main body 5 and serves as identification information reading means, using a high-frequency signal (electromagnetic wave) in a non-contact manner.

An ID read by the RFID reader 62 is inputted to the control section 21. The control section 21 performs flow (rate) control for controlling a process (or processing) of washing/disinfecting while monitoring whether washing and/or disinfecting is being conducted within an appropriate flow rate range and with no clogging in channels, with reference to the ID inputted from the RFID reader 62 and in accordance with channels of the endoscope 2 being washed and disinfected which is contained in the washing and disinfecting bath 3.

The main body 5 is provided with a display section 64 for displaying information on control by the control section 21 or displaying an error. An error may also be indicated with a buzzer instead of being displayed. Alternatively, an error may also be indicated both through sound from a buzzer and display on the display section 64.

FIG. 3 shows a configuration of the flow rate control section 4.

As shown in FIG. 3, the control unit 21 controls ON/OFF operations of the pump 15 and the compressor 22. The control section 21 also controls switching of the change-over valve 16. Specifically, when washing water in the washing and disinfecting bath 3 is supplied to a channel of the endoscope 2, the control section 21 switches the change-over valve 16 so that the valve 16 communicates with the channel 14 on the pump 15 side.

On the other hand, when a channel should be rinsed when washing water being supplied into the channel is changed to disinfectant, washing water in the washing and disinfecting bath 3 is discharged and thereafter the change-over valve 16 is switched so that the valve 16 communicates with the channel 23 on the compressor 22 side.

When washing/disinfecting of the channel has finished and the channel is to be drained or dried, the change-over valve 16 is also switched to communicate with the channel 23 on the compressor 22 side.

After switching by the change-over valve 16, the flow rate of liquid or air is measured by the flow rate sensor 17 and a measured flow rate is inputted to the control section 21.

The control section 21 also uses the ID of the endoscope 2 read by the RFID reader 62 to read information in channels used in the endoscope 2 having that ID, which is stored, for example, in the flash memory 63 which serves as channel information storing section.

In the flash memory 63, channel information including the inner diameter of channels of the endoscope 2 is prestored being associated with, for example, the ID of the endoscope 2. The control section 21 can read out corresponding channel information by specifying an ID, for example, as an address. The flash memory 63 may also be provided inside the control section 21. Alternatively, channel information on the endoscope 2 may be prestored in memory in the RFID tag 61 of the endoscope 2, and the control section 21 may read the channel information through the RFID reader 62.

In accordance with channel information read out, the control section 21 performs determination of whether flow rate is appropriate for washing or disinfecting or whether any channel is clogged or not, and/or control for washing/disinfecting process, e.g., opening/closing of the electromagnetic valves 18a to 18c. For example, for an endoscope that does not have the wire insertion channel 59a, the control section 21 performs control so that a process of washing or disinfecting the wire insertion channel 59a is not performed (in such a case, the electromagnetic valve 18c is left closed).

Also, as to the flow rate sensor 17 used in the present embodiment, a range of measurement is limited with a single flow rate sensor.

For example, if a measurable range is set such that flow rate of the suction channels 57a and 57b, which are large-flow channels, can be measured within an upper limit value of the flow rate, flow rate cannot be measured in the wire insertion channel 59, which is a special channel of an extremely small inner diameter, with a required level of accuracy because flow in the channel 59a is too small.

On the other hand, if a measurable range is set such that flow rate in the wire insertion channel 59a, which is a special channel of an extremely small inner diameter, can be measured, flow rate in gas/water supply channels, which are channels of a medium flow rate (more specifically, the gas supply channels 53a, 53b, and water supply channels 54a, 54b), can be measured within the upper limit value of the measurable range, but flow rate in the suction channels 57a and 57b which are large-flow channels cannot be measured within the upper limit value.

Accordingly, as shown in FIG. 3, the present embodiment inserts an orifice 19a for limiting flow in the channel 14a in which the electromagnetic valve 18a is provided midway and which is connected to the suction channels 57a and 57b, and inserts an orifice 19b in the channel 14b in which the electromagnetic valve 18b is inserted midway and which is connected to the gas supply channels 53a, 53b and the water supply channels 54a, 54b.

Also, in this case, the orifice diameter of the orifice 19a is set to be smaller than that of the orifice 19b.

As a specific example, the orifice diameter of the orifice 19a which is connected in series to the suction channels 57a and 57b, which are large-flow channels, is set to 3 mm, for example, whereas the orifice diameter of the orifice 19b which is connected in series to the gas/water supply channels (the gas supply channels 53a, 53b, and the water supply channels 54a, 54b), which are medium-flow channels, is set to 5 mm.

In the present embodiment, flow in the channel 14a which is connected in series to large-flow channels is limited more by the orifice 19a than the orifice 19b in the channel 14b which is connected in series to medium-flow channels.

Likewise, flow in the channel 14b which is connected in series to medium-flow channels is limited with the orifice 19b so that the flow rate sensor 17 capable of measuring the flow rate of an extremely narrow channel can measure flow rate in a channel of any size, thereby ensuring accuracy of flow rate measurement (i.e., enabling flow rate control that prevents degradation of flow rate measurement accuracy).

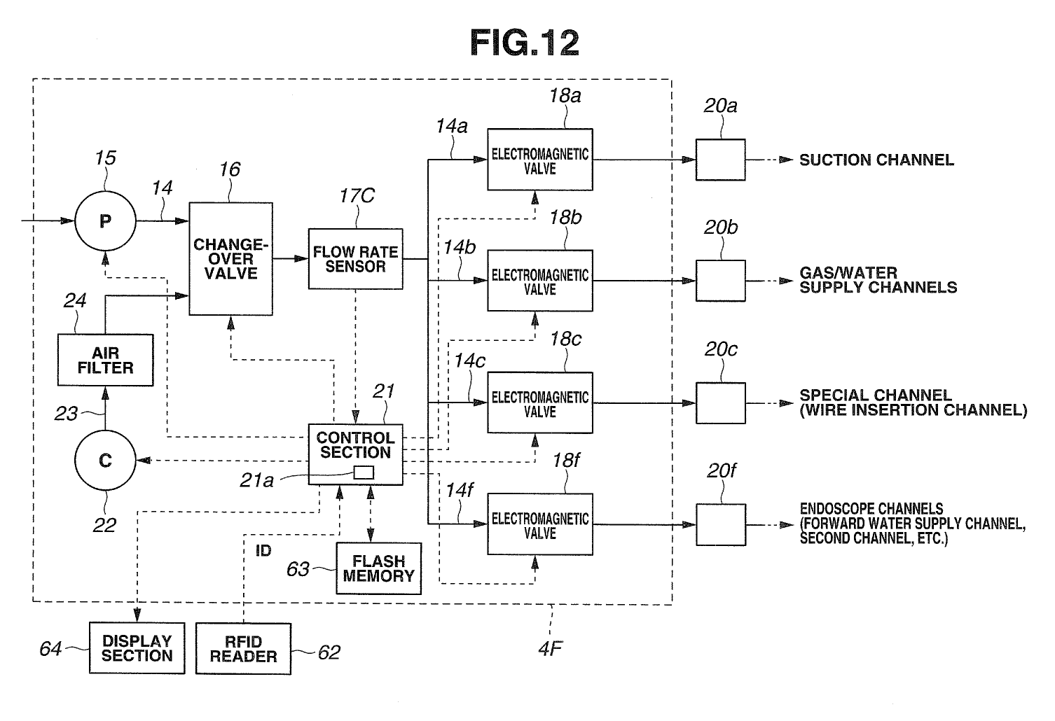

The present embodiment is described with a configuration in which washing water or the like is supplied simultaneously to the gas supply channels 53a, 53b and the water supply channels 54a, 64b among gas/water supply channels, for example. However, a channel similar to the channel 14b (as well as the electromagnetic valve 18b and orifice 19b and the like) may be further provided so that fluid or the like may be supplied to the gas supply channels 53a, 53b and to the water supply channels 54a, 54b with a time difference therebetween (see FIG. 12 for an example of this configuration).

Also, while FIG. 2 shows a case where washing water or the like is supplied to the suction channels 57a and 57b, for example, in parallel for washing or disinfection, washing water or the like may be supplied from the suction mouthpiece 57c into the suction channels 57a and 57b in a serial manner for washing/disinfecting the channels. Other gas supply channels 53a, 53b, and water supply channels 54a, 54b may be washed and disinfected in a similar manner.

Thus, in the present embodiment, when washing water (or liquid) and/or disinfectant (also referred to as washing/disinfecting liquid) is supplied at least by the pump 15 into a plurality of channels of the endoscope 2 for washing and disinfecting thereof, the electromagnetic valves 18a to 18c are provided in the individual channels 14a to 14c serving as connecting channels connected to the plurality of channels of the endoscope. The present embodiment also provides the single flow rate sensor 17 between the pump 15 and the electromagnetic valves 18a and 18c. A characteristic of the present embodiment is the provision of the orifice 19a as a flow rate limiting section for limiting flow into at least the suction channels 57a and 57b that are of the largest inner diameter among the plurality of channels so that flow rate is brought into a range of flow rate measurement in which flow rate measurement by the flow rate sensor 17 is possible.

In other words, a characteristic of the present embodiment is provision of the orifice 19a as a flow rate limiting section for limiting flow into at least the suction channels 57a and 57b which have the largest inner diameter in which fluid such as washing water flows at a flow rate exceeding (the upper limit of) the flow rate measurement range of the flow rate sensor 17 among the plurality of channels, so that flow rate comes within a range of flow rate measurement in which flow rate measurement by the flow rate sensor 17 is possible.

The present embodiment provides the orifice 19b serving as a flow rate limiting section also for flow supplied to gas/water supply channels in order to further improve accuracy of flow rate measurement. When fluid is supplied to the gas/water supply channels, the orifice 19b is not an essential component if the flow rate of the fluid is within the flow rate measurement range of the flow rate sensor 17.

Next, a typical example of processing in a washing and disinfecting process by the endoscope washing and disinfecting apparatus 1 of the present embodiment will be described with reference to FIG. 4.

The user places the endoscope 2 to be washed and disinfected in the washing and disinfecting bath 3 of the endoscope washing and disinfecting apparatus 1 as shown in FIG. 1. When placing the endoscope 2, the user connects the connecting sections of the channels of the endoscope 2 with the suction channel connecting mouthpiece 20a, gas/water supply channel connecting mouthpiece 20b, and special channel connecting mouthpiece 20c of the washing and disinfecting bath 3 via the connecting tubes 37a, 37b, and 37c, respectively.

The user then powers on the endoscope washing and disinfecting apparatus 1 to start operations of washing and disinfecting process as shown at step S1 of FIG. 4.

At the first step S1, the control section 21 in the main body 5 starts control operations in accordance with a control program written, for example, in the flash memory 63, and performs processing for obtaining channel information of the endoscope.

Specifically, the control section 21 issues an instruction for reading ID information in the RFID tag 61 to the RFID reader 62. Upon receiving the instruction, the RFID reader 62 sends a signal for reading an ID to the RFID tag 61 and has the tag 61 send ID information.

The RFID reader 62 sends the obtained ID information to the control section 21.

The control section 21 uses the inputted ID to read channel information of the endoscope 2, which is placed in the washing and disinfecting bath 3, from the flash memory 63 to obtain channel information.

As shown at the following step S2, the control section 21 recognizes from the channel information that the endoscope 2 in the washing and disinfecting bath 3 is an endoscope 2 that has the suction channel 57a, gas supply channel 53a and water supply channel 54a, and the wire insertion channel 59a as a special channel. The control section 21 also recognizes from the channel information an appropriate flow rate range for each channel in a case where liquid is supplied to the channel using the pump 15 of the present embodiment.

In the present embodiment, the control section 21 also recognizes an appropriate flow rate range in the suction channels 57a, 57b and the gas/water supply channels in a case where flow rate is limited using the orifices 19a and 19b.

At the following step S3, the control section 21 controls various sections of the main body 5 to supply washing water into the suction channels 57a, 57b, the gas/water supply channels (i.e., gas supply channels 53a, 53b, and water supply channels 54a, 54b), and the wire insertion channel 59a in sequence to start a washing process.

In this case, the control section 21 periodically has the flow rate sensor 17 measure flow rate and obtains a measured flow rate as shown at step S4.

In this case, since the present embodiment limits flow in a channel of a large inner diameter (which results in a high flow rate), the single flow rate sensor 17 can measure the flow rate of respective channels with high accuracy even when the channels have varying inner diameters.

As shown at the following step S5, the control section 21 determines whether the flow rate measured by the flow rate sensor 17 is proper or not. If it determines that the detected flow rate is within a proper flow rate range, the control section 21 continues the washing process.

However, if it determines that the detected flow rate is not within a proper flow rate range, the control section 21 displays an error indicating that the detected flow rate is not within a proper flow rate range on, for example, the display section 64 as shown at step S6, and terminates the washing and disinfecting process of FIG. 4.

When the washing process has terminated with the flow rate determined to be proper, the control section 21 starts a rinsing process as shown at step S7. In this case, washing water in the washing and disinfecting bath 3 is first drained. Thereafter, the control section 21 switches the change-over valve 16 so that the valve 16 communicates with the channel 23 on the side of the compressor 22, and sequentially supplies air to channels of the endoscope 2 with the compressor 22.

Also in this case, as shown at step S8, the control section 21 periodically has the flow rate sensor 17 measure a flow rate and obtains a measured flow rate. Then, as shown at step S9, the control section 21 determines whether the measured flow rate is proper or not. That is to say, the control section 21 has functions as a flow rate determining section for determining whether a measured flow rate is proper or not.

If it determines that the detected flow rate is within a proper flow rate range, the control section 21 continues the rinsing process.

On the other hand, if it determines that the detected flow rate is not within a proper flow rate range, the control section 21 displays an error indicating that the detected flow rate is not within a proper flow rate range on, for example, the display section 64 as shown at step S6, and terminates the washing and disinfecting process of FIG. 4.

When the rinsing process has terminated with the flow rate determined to be proper, the control section 21 starts a disinfection process as shown at step S10.

In this case, disinfectant in the disinfectant tank 32 is supplied into the washing and disinfecting bath 3, and disinfectant supplied into the washing and disinfecting bath 3 is taken into the channel 14 and supplied to each channel of the endoscope 2 with the pump 15.

Also in this process, flow rate is periodically measured according to step S11, and determination is made as to whether a measured flow rate is proper or not as shown at step S12, and an error is displayed or otherwise indicated at step S6 if the flow rate is not within a proper range. On the other hand, if the measured flow rate is proper, the disinfection process is continued.

When the disinfection process finishes, the rinsing process at step S13 is carried out.

In a first half of the rinsing process, disinfectant in the washing and disinfecting bath 3 is collected into the disinfectant tank 32 and/or, if the disinfectant is unclean, the drain pump 30 is run to drain the disinfectant.

Thereafter, the compressor 22 is operated to supply air as in the rinsing process at step S7. In this process, flow rate is also periodically measured at step S14, and determination is made as to whether a measured flow rate is proper or not as shown at step S15, and an error is displayed or otherwise indicated at step S6 if it is not within a proper flow rate range. Meanwhile, if the measured flow rate is a proper flow rate, the rinsing process is continued. When the rinsing process is complete, a draining process at step S16 (or a draining and air supplying process) is performed.

In this case, after the rinsing process, air is further supplied into channels to dry the channels. In this case, the electromagnetic valves 18a to 18c may be sequentially opened and closed, or simultaneously opened and closed.

After sufficient drainage, the washing and disinfecting process finishes. The operation example shown in FIG. 4 is merely an example and not restrictive.

As has been described, according to the present embodiment, even when the endoscope 2 having a plurality of channels of different inner diameters is washed and disinfected, flow rate control with accurate detection of flow rate in any of the channels is possible with a single flow rate sensor 17 because flow rate limiting means is provided that limits flow in a channel that is of a large inner diameter and thus has a high flow rate.

Therefore, by measuring flow rate, it is possible to accurately determine whether each process in the washing and disinfecting process is being performed with an appropriate flow rate. In addition, by performing washing and disinfecting with a proper flow rate, it is possible to ensure quality of processing for washing and disinfecting.

In addition, by enabling determination of whether flow rate is proper or not, washing and disinfecting can be efficiently performed through control for automatically continuing a washing and disinfecting process if such determination shows that the flow rate is proper.

Additionally, according to the present embodiment, since only one flow rate sensor 17 is required, it is possible to realize the endoscope washing and disinfecting apparatus 1 that conducts washing and disinfecting efficiently and at a low cost.

Although the present embodiment is shown with a configuration in which flow is also limited when liquid is supplied into gas/water supply channels that are of smaller inner diameters in addition to when liquid is supplied to the suction channels 57a and 57b of the largest inner diameter, flow may be limited only when liquid is supplied into a channel of the largest inner diameter, as a variation of this configuration.

### Second Embodiment

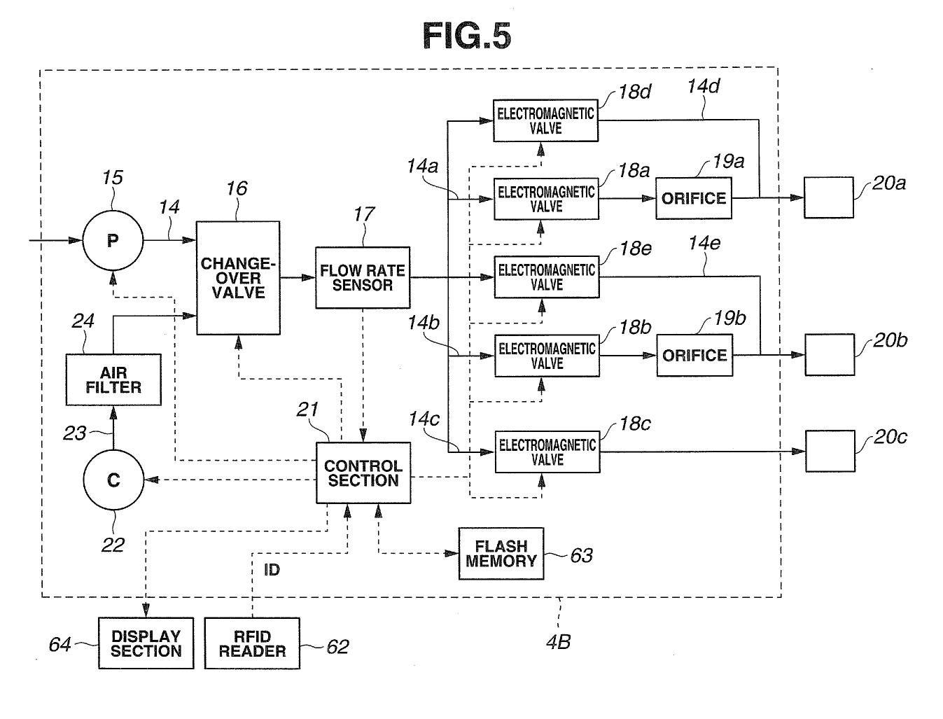

FIG. 5 shows a configuration of a flow rate control section 4B according to a second embodiment of the present invention. The endoscope washing and disinfecting apparatus according to the present embodiment has a configuration in which the flow rate control section 4 is replaced with the flow rate control section 4B shown in FIG. 5 in the endoscope washing and disinfecting apparatus 1 of FIG. 1.

The flow rate control section 4B shown in FIG. 5 provides a channel 14d as a bypass connecting channel (or a bypass channel) which is parallel with the electromagnetic valve 18a and the orifice 19a, and also an electromagnetic valve 18d for opening and closing the channel 14d midway in the channel 14d, to the flow rate control section 4 shown in FIG. 3. That is to say, the flow rate control section 4B has the channel 14d which is parallel with the channel 14a in which the electromagnetic valve 18a and orifice 19a are provided. When the electromagnetic valve 18d inserted in the channel 14d is opened by the control section 21, the channel 14d has functions as a bypass channel used as a bypass.

The flow rate control section 4B similarly has a channel 14e as a bypass connecting channel (or a bypass channel) which is parallel with the electromagnetic valve 18b and orifice 19b, and is provided with an electromagnetic valve 18e for opening and closing the channel 14e midway in the channel 14e.

In the present embodiment, the channel 14e is provided in parallel with the channel 14b in which the electromagnetic valve 18b and orifice 19b are provided, and when the electromagnetic valve 18e inserted in the channel 14e is opened, the channel 14e has functions as a bypass channel.

The control section 21 controls opening and closing of the electromagnetic valves 18a to 18c as well as electromagnetic valves 18d and 18e.

More specifically, when controlling opening/closing of the electromagnetic valves 18d and 18e, the control section 21 basically opens or closes the valves 18d and 18e in conjunction with opening/closing of the electromagnetic valves 18a and 18b in the first embodiment. However, during a period in which flow rate is measured or detected by the flow rate sensor 17, the electromagnetic valves 18d and 18e are closed to allow measurement of flow rate. The configuration is otherwise similar to that of the first embodiment.

In the first embodiment, flow is limited or reduced to bring down a high flow rate to a lower flow rate so as to enable measurement with the flow rate sensor 17. However, the present embodiment reduces flow (in a channel in which flow rate measurement is impossible unless flow is reduced) only at the time of flow rate measurement and does not reduce flow in a period when flow rate measurement is not performed.

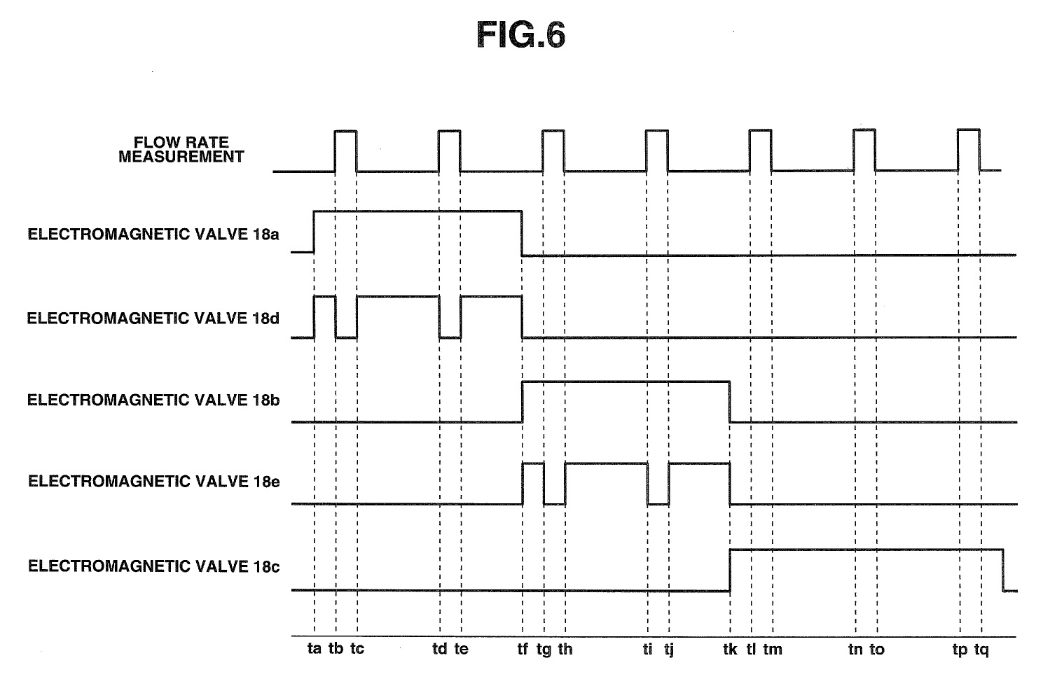

FIG. 6 illustrates operations according to the present embodiment. FIG. 6 shows control by the control section 21 for opening/closing the electromagnetic valves 18a to 18e during, for example, a washing process of a washing and disinfecting process in the present embodiment.

As described in the first embodiment, in a washing process, flow rate is periodically measured, for example. In the present embodiment as well, the control section 21 obtains a measured value of flow rate measured by the flow rate sensor 17 in time periods, tb-tc, td-te, tg-th, ti-tj, tl-tm, tn-to, and tp-tq, for example, during a washing process as shown in FIG. 6.

Also, when a washing process starts, the electromagnetic valves 18a and 18d are switched from being close to open at time, ta, for example, as shown in FIG. 6. Then, a washing process for the suction channels 57a and 57b starts. During time, ta-tf, in the washing process for the suction channels 57a and 57b, the electromagnetic valve 18d is closed during times of flow rate measurement, tb-tc and td-te.

When the washing process for the suction channels 57a and 57b performed in such a way finishes, the electromagnetic valves 18a and 18d are closed.

When the washing process for the suction channels 57a and 57b finishes, the electromagnetic valves 18b and 18e are switched from close to open, and a washing process for gas/water supply channels (gas supply channels 53a, 53b, and water supply channels 54a, 54b) starts. During time, tf-tk, in the washing process for the gas/water supply channels, the electromagnetic valve 18e is closed during times of flow rate measurement, tg-th and ti-tj.

Thus, when the washing process for the gas/water supply channels finishes, the electromagnetic valves 18b and 18e are closed.

After the washing process for the gas/water supply channels finished, the electromagnetic valve 18c is switched from close to open, and a washing process for the wire insertion channel 59a as a special channel starts. During the time of the washing process for the wire insertion channel 59a, the electromagnetic valve 18c is left open all the time.

Then, when the washing process for the wire insertion channel 59a finishes, the electromagnetic valve 18c is closed.

Then, the following rinsing process is entered. In the rinsing process, the electromagnetic valves 18a to 18e are also controlled in a similar manner. The electromagnetic valves 18a to 18e are similarly controlled in other processes after the rinsing process as well.

According to the present embodiment, washing/disinfecting is carried out with reduction of flow in a channel for which flow rate cannot be measured unless flow is reduced or limited only during a time (period) of flow rate measurement so as to enable flow rate measurement and without reducing flow during a period in which flow rate measurement is not performed. Therefore, the present embodiment can complete processing for the washing or disinfection process in a smaller amount of time than the first embodiment. The present embodiment otherwise has similar advantages as those of the first embodiment.

While this embodiment is shown as an application to the configuration of FIG. 3, it may also be applied to a configuration in which flow is limited only when liquid is supplied to the suction channels 57a and 57b, which are of the largest inner diameter, as a variation of FIG. 3, for example.

In the first and second embodiments, when fluid is supplied into a plurality of channels of the endoscope 2 and the flow rate of the fluid is measured with the single flow rate sensor 17, flow in a channel that exceeds the upper limit value of a flow rate measurement range is limited to be brought into the flow rate measurement range that can be measured by the flow rate sensor 17.

Meanwhile, a third embodiment discussed below pads flow rate in a channel having a flow rate that does not reach a lower limit value of the flow rate measurement range of the flow rate sensor 17 so that it falls within the flow rate measurement range that can be measured by the flow rate sensor 17.

### Third Embodiment

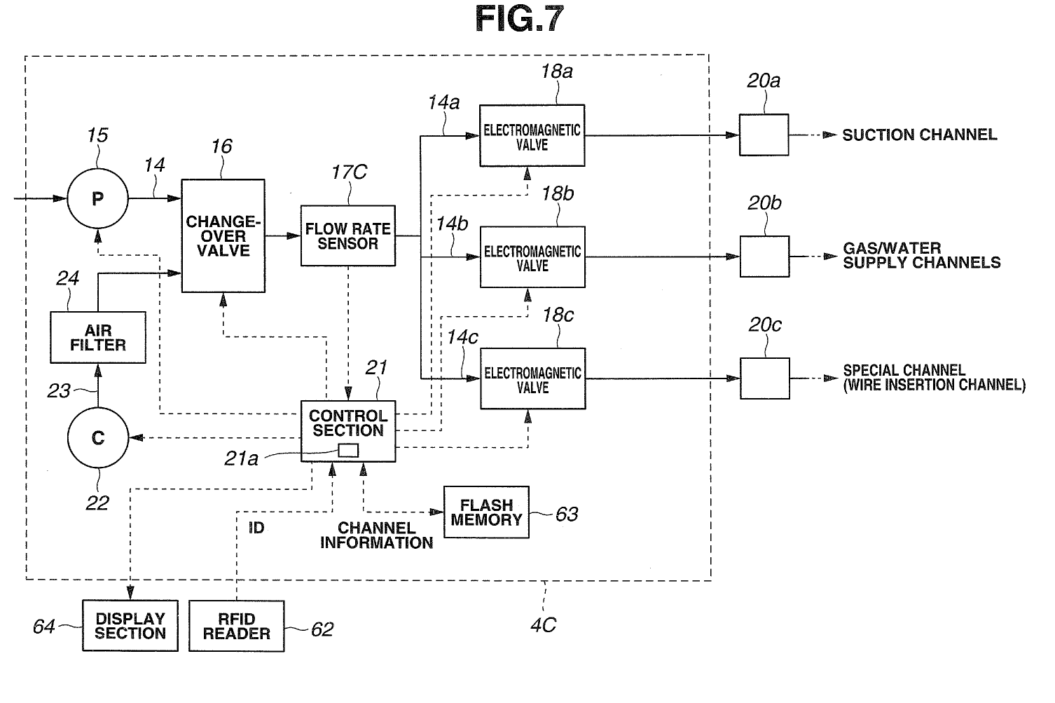

FIG. 7 shows a configuration of a flow rate control section 4C according to a third embodiment of the invention. The endoscope washing and disinfecting apparatus of the present embodiment has a configuration in which the flow rate control section 4 of the endoscope washing and disinfecting apparatus 1 of FIG. 1 is replaced with the flow rate control section 4C shown in FIG. 7.

The flow rate control section 4C shown in FIG. 7 has a configuration that does not include the orifices 19a and 19b of the flow rate control section 4 of FIG. 3. Also, the present embodiment adopts a flow rate sensor 17C capable of measuring a high flow rate in place of the flow rate sensor 17 of the first embodiment.

Also, in the present embodiment, when measuring flow rate in a special channel with the electromagnetic valve 18c open, the control section 21 performs flow rate padding control by adding a flow rate that can be calculated within the measurement range of the flow rate sensor 17C (to be specific, adding a flow rate in the suction channels 57a and 57b with the electromagnetic valve 18a open in the channel 14a, which is connected to the suction channels 57a and 57b).

More specifically, when liquid is supplied to a special channel of a small inner diameter, its flow rate is too small to be measured by the flow rate sensor 17C with a required accuracy. Therefore, an offset value that can be calculated is added to bring the value into a flow measurement range in which measurement is possible so as to enable measurement with the flow rate sensor 17C. Then, after obtaining a measured value with the offset value added, the control section 21 calculates a net flow rate in a case where liquid is supplied to the special channel, by performing an operation of subtracting the offset value.

The control section 21 therefore includes a control function 21a of a flow rate padding section for padding flow rate with an offset flow rate value so that the flow rate comes within a range that can be measured by the flow rate sensor 17C, when flow rate is measured in a special channel in which flow rate is too small and fluid flows at a flow rate that falls short of the lower limit value of the flow measurement range.

For a flow rate used as the offset value, namely a flow rate that can be calculated, a flow rate in a suction channel or a flow rate in gas/water supply channels is adopted, for example.

Because the flow rate to be flown in the suction channels 57a and 57b or the gas/water supply channels which is used as the offset value can be obtained through actual measurement by the flow rate sensor 17 when liquid is not supplied to the special channel, it is possible to easily perform processing for detecting a net flow rate in a case where liquid is supplied to the special channel.

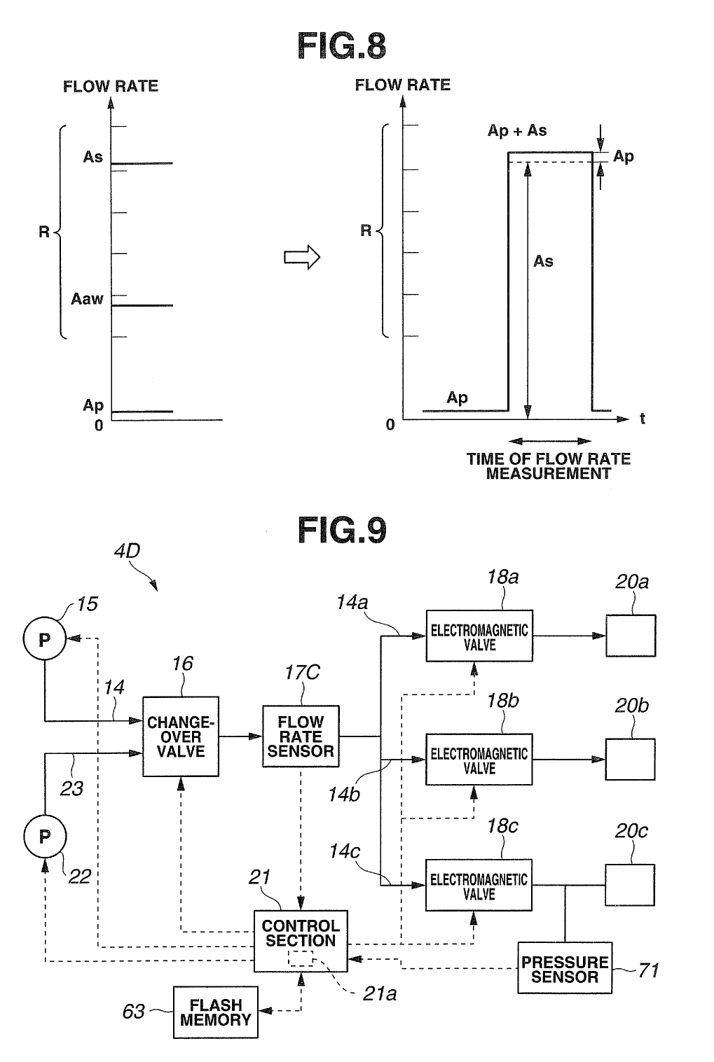

FIG. 8 shows a diagram illustrating operations in the present embodiment. A diagram in a left portion of FIG. 8 approximately shows a flow rate measurement range R that can be measured by the flow rate sensor 17C, where flow rate, As, of the suction channels 57a and 57b as well as the flow rate, Aaw, of the gas/water supply channels fall within the flow measurement range R. However, the flow rate, Ap, of a special channel having a very small effective inner diameter, such as the wire insertion channel 59a, is too small and does not reach the flow measurement range R.

Accordingly, as shown at a right portion, when measuring the flow rate, Ap, of a special channel of the smallest inner diameter, such as the wire insertion channel 59a, the control section 21 opens the electromagnetic valve 18a, for example, to supply liquid also to the suction channels 57a and 57b. The present embodiment thereby pads the flow rate, Ap, of a special channel, such as the wire insertion channel 59a, which is to be measured by the flow rate sensor 17C, to Ap+As.

Then, after obtaining the padded flow rate (from the flow rate sensor 17C), the control section 21 subtracts the flow rate, As, of the suction channels 57a and 57b to calculate the flow rate, Ap, of the special channel.

The present embodiment has an advantage of measuring the flow rate, Ap, of a special channel having a too small flow rate with fewer components than the first or second embodiment.

FIG. 9 shows a configuration of a flow rate control section 4D in a first variation of the present embodiment. The configuration of the flow rate control section 4D adds a pressure sensor 71 for detecting pressure in the channel 14c which leads from the electromagnetic valve 18c to the special channel connecting mouthpiece 20c, to the flow rate control section 4C shown in FIG. 7. Note that the air filter 24 is omitted in FIG. 9 (and FIG. 11 discussed below) for the sake of simplicity.

In the present variation, flow rates in the suction channels 57a, 57b, and the gas/water supply channels are measured with the flow rate sensor 17C described above. For a special channel with a too small flow rate, the flow rate can be measured by padding it as described above or the measurement thereof may be omitted. As the control function 21a of the flow rate padding section in FIG. 9 is shown by a dotted line because it may be either used or not.

And using the pressure sensor 71, the degree of clogging in the special channel is detected with high accuracy from change in pressure of the special channel.

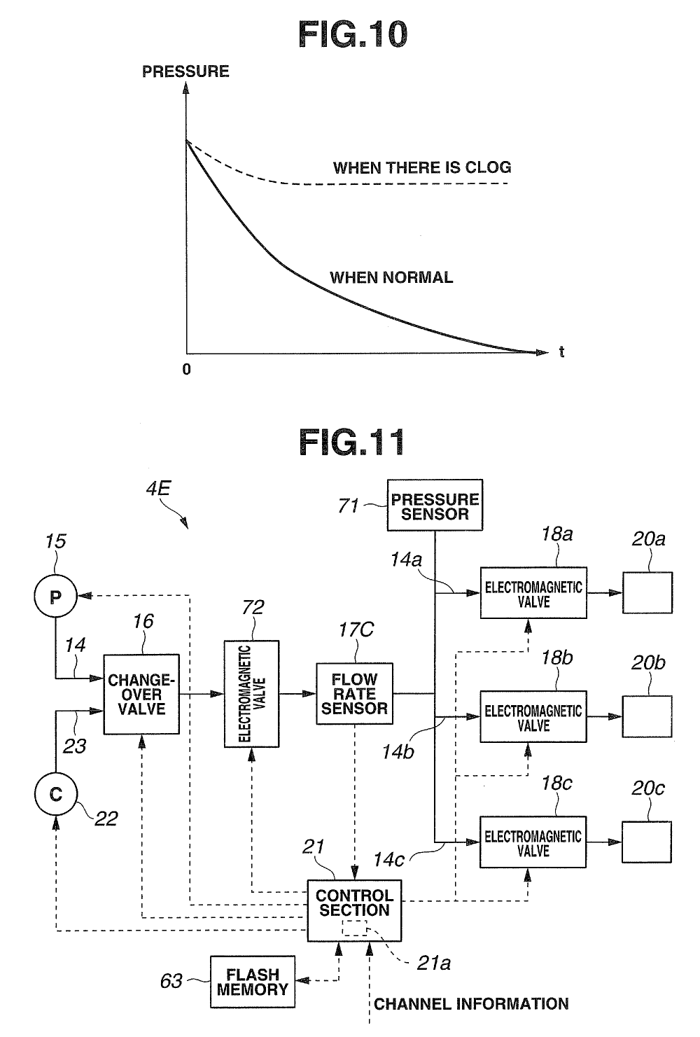

Specifically, when liquid or air has been supplied into the special channel with the electromagnetic valve 18c switched from close to open by the control section 21, the electromagnetic valve 18c is closed. The degree of clogging of the special channel is detected based on temporal change in pressure as detected or measured by the pressure sensor 71 from the time at which the electromagnetic valve 18c is closed. Change in pressure in this case is illustrated in FIG. 10.

As shown in FIG. 10, when the special channel is not clogged and in a normal condition, detected pressure lowers with elapse of time, t, as shown by a solid line.

On the other hand, when the special channel is clogged, detected pressure does not lower or lowers less over time as shown by a dotted line. From the trend of pressure change, whether the special channel is clogged or not, and/or degree of clogging can be accurately detected.

According to the present variation, even for a channel with a too low flow rate to be measured with the flow rate sensor 17C, by using the pressure sensor 71 as a pressure gauge, it is possible to detect whether the channel is clogged or not and/or degree of clogging with high accuracy.

FIG. 11 shows a configuration of a flow rate control section 4E according to a second variation of the present embodiment. The flow rate control section 4E has a configuration that adds an electromagnetic valve 72 in the channel 14 on an upstream (or input) side of the flow rate sensor 17C, and a pressure sensor 71 for detecting pressure in the channel 14 on an output side of the flow rate sensor 17C and before the electromagnetic valves 18a to 18c, to the flow rate control section 4C shown in FIG. 7.

In other words, the configuration positions the pressure sensor 71 between the electromagnetic valve 72 and the electromagnetic valves 18a to 18c which are in series with the electromagnetic valve 72.

While the first variation is configured to detect degree of clogging in only a special channel based on change in pressure, the present variation enables measurement of degree of clogging from change in pressure for all of the suction channel, gas/water supply channels, and the special channel.

For example, to detect clogging of a special channel by measuring pressure, after switching the electromagnetic valves 72 and 18c from close to open, the electromagnetic valve 72 is closed and change in pressure is measured by the pressure sensor 71 as in the first variation. In this case, other electromagnetic valves, 18a and 18b, are left closed. By modifying opening/closing control for the electromagnetic valve 18c in this case, existence/absence of clogging or the like of other channels can be measured in a similar way.

In the present variation, the control section 21 decides a combination of flow rate measurement and/or pressure measurement appropriate for a channel recognized from channel information of the endoscope 2, as described in the first embodiment.

The present variation provides a wider choice of detection of an appropriate flow rate range or clogging based on flow rate measurement or detection of clogging based on pressure measurement than the first variation, enabling measurement of a channel flow rate and/or detection of clogging of a channel with higher accuracy even when the endoscope 2 has channels of different types.

FIG. 12 shows a configuration of a flow rate control section 4F in a third variation of the present embodiment, for example. This third variation may be applied to the first or second embodiment.

This variation has a configuration in which a channel 14f as a fourth connecting channel that branches from the channel 14 is provided, and an electromagnetic valve 18f is provided in the channel 14f and an endoscope channel connecting mouthpiece 20f is provided at an end of the channel 14f, in FIG. 7, for example. Opening/closing of the electromagnetic valve 18f is controlled by the control section 21.

For an endoscope having a forward water supply channel in which supplies water forward, for example, the endoscope channel connecting mouthpiece 20f is connected with the forward water supply channel via a connecting tube not shown.

The present variation enables measurement of flow rate or the like also in the forward water supply channel at the time of washing and disinfecting.

The present variation is not limited to a forward water supply channel: for an endoscope having two treatment instrument channels, for instance, the suction channel connecting mouthpiece 20a, for example, is used for a suction channel that communicates with one of the treatment instrument channels as in the above-described embodiment.

Meanwhile, for the second treatment instrument channel, the endoscope channel connecting mouthpiece 20f is connected to a treatment instrument insertion port of the channel via a connecting tube, and the treatment instrument channel can be washed and disinfected just like other channels and flow rate therein can be measured at the time.

In addition, while the above-mentioned embodiments and variations are described with examples where a gas supply channel and a water supply channel are concurrently washed or disinfected, for the endoscope 2 shown in FIG. 2, for example, the connecting mouthpiece 20b and the connecting mouthpiece 20f may be connected to the gas supply channels 53a, 53b, and the water supply channels 54a, 54b of the endoscope 2 using separate connecting tubes.

According to the present variation, even an endoscope having more channels of different types can be appropriately handled at the time of washing and disinfecting. The present variation has otherwise similar advantages to those of the third embodiment. When applied to other embodiment or the like, the present variation also has similar advantages to that embodiment or the like.

### Fourth Embodiment

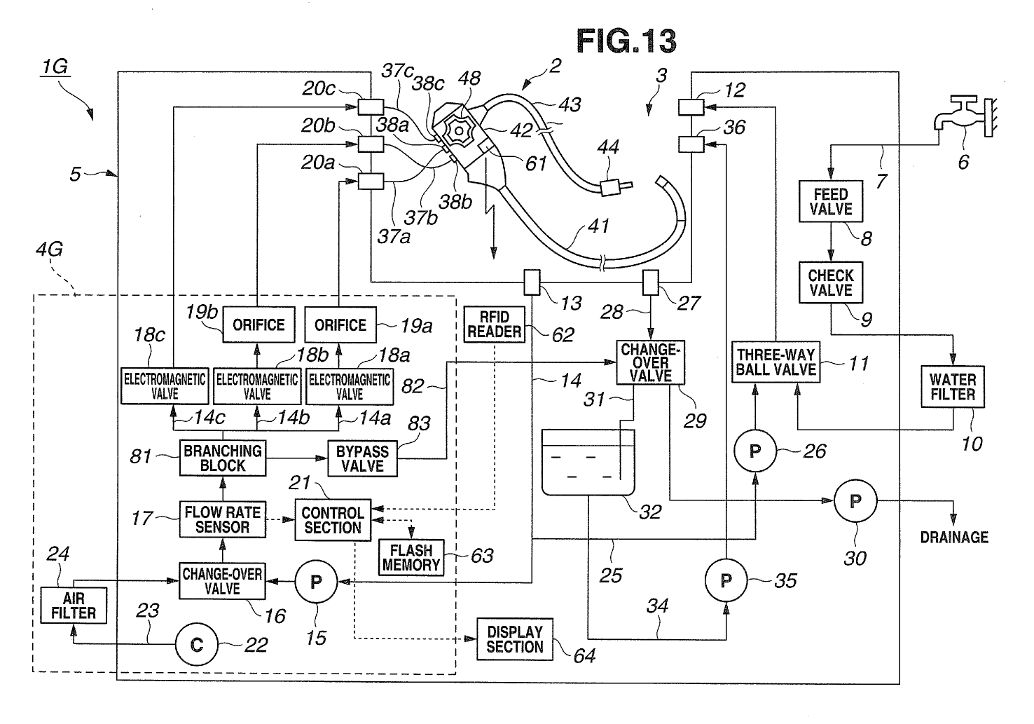

FIG. 13 shows an endoscope washing and disinfecting apparatus 1G according to a fourth embodiment. The endoscope washing and disinfecting apparatus 1G provides a branching block 81 between the flow rate sensor 17 and the electromagnetic valves 18a to 18c in, for example, the endoscope washing and disinfecting apparatus 1 of FIG. 1, and connects a branched channel 82 that branches at the branching block 81 to, for example, the change-over valve 29 with a bypass valve 83 positioned in midway of the channel 82.

The control section 21 constituting a flow rate control section 4F in the present embodiment is allowed to make a first choice for detecting or measuring flow rate in the side of the channels of the endoscope 2 or a second choice for detecting flow rate on the side of the branched channel 82 with the flow rate sensor 17, by switching the branching block 81.

That is, when the first choice is made to switch the branching block 81 so that the flow rate sensor 17 communicates with the electromagnetic valves 18a to 18c side, the configuration and operations are similar to those of the first embodiment.

On the other hand, by making the second choice to switch the branching block 81 so that the flow rate sensor 17 communicates with the side of the branched channel 82 in which the bypass valve 83 is provided, the liquid supply rate of the pump 15 or air supplying rate from the compressor 22 can be measured.

By adding such a simple configuration, the ability of liquid sending or supply by the pump 15 and the ability of the compressor 22 to supply air can be checked under a certain condition near a released condition freed from channels of the endoscope 2 as a load side.

For example, to measure the liquid supplying ability of the pump 15, washing water in the washing and disinfecting bath 3 is supplied to the flow rate sensor 17 side through the channel 14 and guided to the change-over valve 29 via the branched channel 82 which is opened from the branching block 81, and is drained with the change-over valve 29 switched to the drain pump 30 side.

To measure the air supplying ability of the compressor 22, air supplied from the compressor 22 is supplied to the flow rate sensor 17 side and guided to the change-over valve 29 via the branched channel 82 which is opened from the branching block 81, and discharged with the change-over valve 29 switched to the drain pump 30 side. While the above-described configuration connects an end of the branched channel 82 to the change-over valve 29, the present embodiment is not limited thereto. For example, an end of the branched channel 82 may be positioned on the upper surface of the washing and disinfecting bath 3 so that supplied liquid is brought back into the washing and disinfecting bath 3, or supplied air may be discharged to the outside.

According to the present embodiment, a flow rate measuring section is formed that is capable of measuring the ability of the pump 15 and compressor 22 in a certain condition or state with no load or near a released condition without being affected by load which is set to send liquid to each channel of the endoscope 2 during washing or disinfection of the endoscope 2.

Therefore, by providing the branched channel 82 and measuring the flow rate of fluid flowing in the channel 82, degradation or the like of pumps as fluid sources can be grasped with high accuracy. The present embodiment otherwise has similar advantages to those of the first embodiment.

For the compressor 22, measurement of its pressure may be allowed so that temporal change in characteristics of the compressor 22 or the like can be detected from pressure.

### Fifth Embodiment

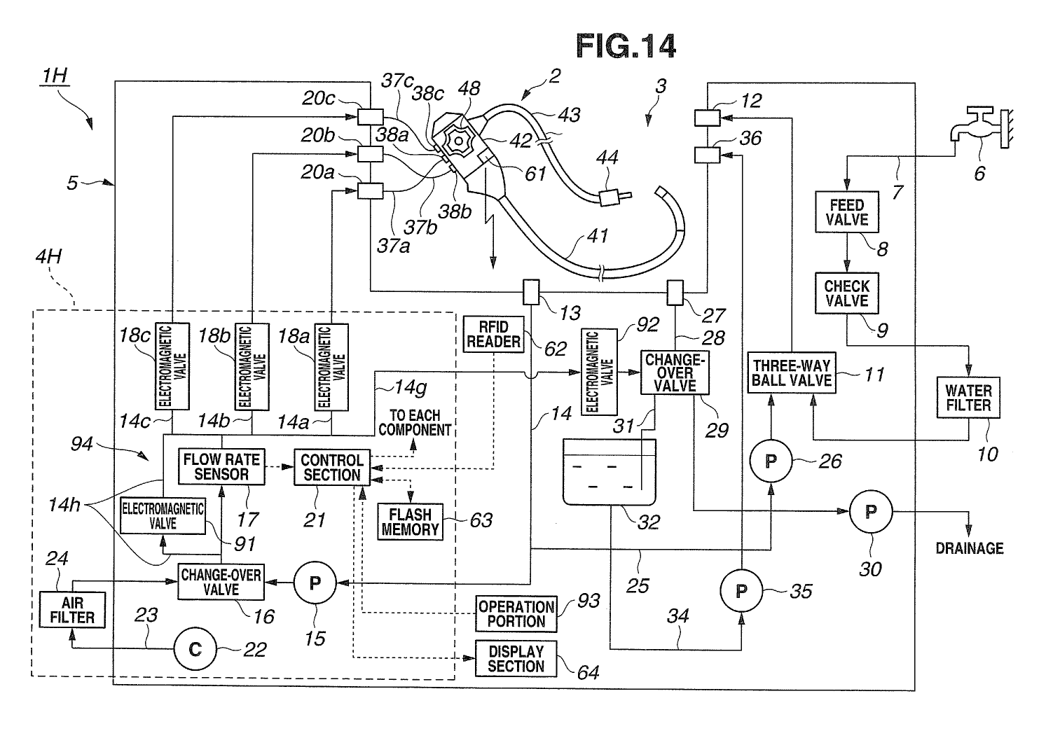

Next, referring to FIG. 14, an endoscope washing and disinfecting apparatus 1H according to a fifth embodiment of the invention is described. The endoscope washing and disinfecting apparatus 1H has a configuration that does not include the two orifices 19a and 19b that form a flow rate limiting section in the endoscope washing and disinfecting apparatus 1 of the first embodiment shown in FIG. 1, for example, and adopts a flow rate control section 4H of a configuration with two electromagnetic valves 91 and 92. Opening/closing operation of the two electromagnetic valves 91 and 92 is controlled by the control section 21.

One of the electromagnetic valves, 91, is inserted in a bypass channel 14h which is parallel with the flow rate sensor 17 positioned between the change-over valve 16 and the electromagnetic valves 18a to 18c. In other words, the electromagnetic valve 91 is positioned in the bypass channel 14h which communicates the input side of the flow rate sensor 17 with the output side thereof in the channel 14 in which the flow rate sensor 17 is inserted. While in FIG. 14 one end of the bypass channel 14h is designed to branch midway of the channel 14 which leads from the change-over valve 16 to the flow rate sensor 17, the end may branch directly from the change-over valve 16.

And by opening and closing the electromagnetic valve 91, flow rate to the flow rate sensor 17 can be changed or adjusted.

For example, when the electromagnetic valve 91 is closed, a flow rate equal to that in a case where the electromagnetic valve 91 is not provided flows through the flow rate sensor 17. On the other hand, when the electromagnetic valve 91 is opened, a flow rate of flow from the side of the change-over valve 16 divides into a flow rate that flows to the flow rate sensor 17 and a flow rate that flows to the bypass channel 14h. Therefore, the flow rate that flows to the flow rate sensor 17 is smaller than when the electromagnetic valve 91 is closed.

As the flow rate sensor 17, the present embodiment employs a flow rate sensor capable of measuring a flow rate in an extremely narrow channel (specifically, a special channel such as a wire insertion channel) within its flow rate measurement range, as described in the first embodiment.

Also, the inner diameter or the like of the bypass channel 14h and electromagnetic valve 91 is appropriately configured so that the flow rate that flows to the flow rate sensor 17 side can be measured within the flow rate measurement range by opening the electromagnetic valve 91 for a large-flow channel (specifically, a suction channel). In addition, from the flow rate on the flow rate sensor 17 side, a flow rate that flows on the side of the bypass channel 14h of a certain inner diameter with the electromagnetic valve 91 open can be known.

Flow rate in a medium-flow channel (specifically, gas/water supply channels) can be measured by the flow rate sensor 17 with the electromagnetic valve 91 either open or closed. Example operations below will be described with an example where the electromagnetic valve 91 is open.

The other electromagnetic valve 92 is positioned midway in a channel 14g which communicates with a channel on the output side of the flow rate sensor 17 and leads to the change-over valve 29. With the channel 14g provided with the electromagnetic valve 92 which is opened or closed through control, a liquid supply rate as the liquid supplying ability of the pump 15 itself (alone), which constitutes a fluid supply unit, can be measured. Hereinafter, the liquid supply rate of the pump 15 itself will be referred to as just a liquid supply rate of a pump or a liquid supply rate of the pump 15. The control section 21 stores a measured liquid supply rate and uses the rate to determine whether flow rates in various channels in the endoscope 2 are proper or not with high accuracy.

Thus, for example, when the operator performs an instructive operation for measuring and storing the liquid supply rate of the pump 15 itself from the operation portion 93 provided on the main body 5, which serves as instructive operation means, to the control section 21, the control section 21 measures the liquid supply rate of the pump 15 and stores the rate in the flash memory 63 as described below.

The flash memory 63 has also prestored therein information for setting a flow rate threshold value used for determining that no channel of the endoscope 2 is clogged and flow rate is within a proper range or that any channel is clogged based on a measured value of liquid supply rate of the pump 15. For example, the control section 21 calculates a threshold value for determining a proper flow rate range and a condition with clogging with a calculation formula, e.g., from information on diameter of various channels of the endoscope 2 and stores the threshold value in the flash memory 63.

The control section 21 then compares a measured value of flow rate in a case where liquid is actually supplied to one of various channels with the threshold value to determine whether there is clogging in that channel or not. The threshold value used for determining whether there is clogging is not limited to a single value but a number of threshold values may be set depending on degree of clogging. In addition, instead of information on a threshold for determining occurrence of clogging, information on a proper flow rate range may be stored in the flash memory 63 in combination with threshold value information.

Thus, the present embodiment does not provide the orifices 19a and 19b that constitute flow rate limiting sections. And the present embodiment provides a flow rate diverting section 94 that enables the bypass channel 14h provided in parallel with the flow rate sensor 17 to be opened and closed through the electromagnetic valve 91 to limit the flow rate that flows into the flow rate sensor 17 to within the flow rate measurement range of the flow rate sensor 17, and diverts a portion of flow that exceeds the flow rate measurement range through the bypass channel 14h.

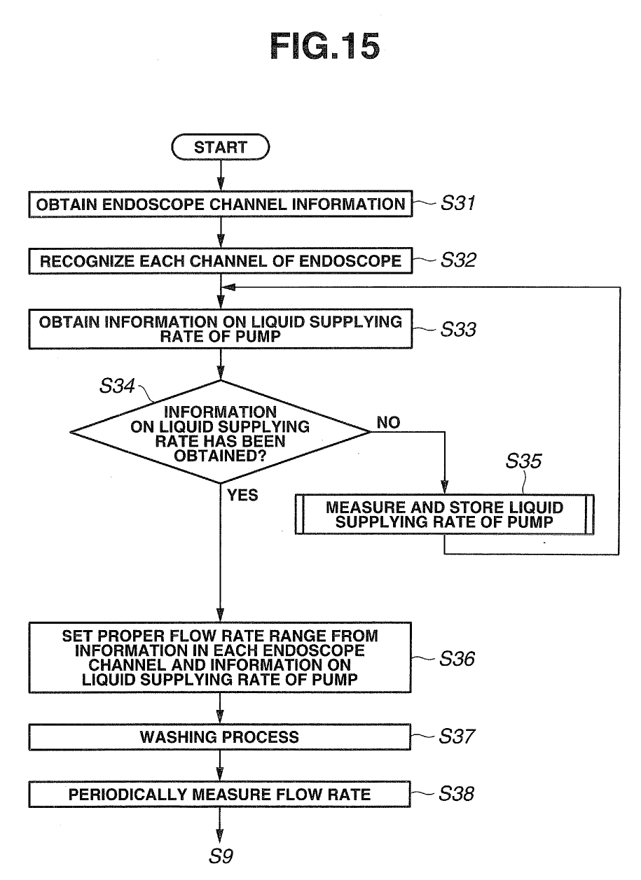

The configuration is otherwise similar to that of the first embodiment. Next, operations of the present embodiment having such a configuration will be described with reference to FIG. 15. FIG. 15 shows a part of a process of washing/disinfecting the endoscope 2 performed by the endoscope washing and disinfecting apparatus 1H of the present embodiment. Overall processing in this case is almost the same as what was described in FIG. 4.

A user places the endoscope 2 to be washed and disinfected in the washing and disinfecting bath 3 of the endoscope washing and disinfecting apparatus 1 as shown in FIG. 14. In this case, the user connects the connecting sections of channels of the endoscope 2 with the suction channel connecting mouthpiece 20a, gas/water supply channel connecting mouthpiece 20b, and special channel connecting mouthpiece 20c of the washing and disinfecting bath 3 via the connecting tubes 37a, 37b and 37c, respectively.

The user then powers on the endoscope washing and disinfecting apparatus 1H and starts operations of the washing and disinfecting process as shown at step S31 of FIG. 15.

At the first step S31, the control section 21 in the main body 5 starts control operations in accordance with a control program written, for example, in the flash memory 63, and performs processing for obtaining channel information of the endoscope 2.

Specifically, the control section 21 uses the RFID reader 62 to obtain ID information in the RFID tag 61 on the endoscope 2. The control section 21 then uses the ID information to obtain channel information of the endoscope 2, which is being contained in the washing and disinfecting bath 3, from the flash memory 63.

Then, as shown at step S32, the control section 21 recognizes from the channel information that the endoscope 2 in the washing and disinfecting bath 3 is an endoscope 2 that has the suction channel 57a, gas supply channel 53a and water supply channel 54a, and the wire insertion channel 59a as a special channel. In the present embodiment, in addition to the channel information, the control section 21 obtains information on the liquid supply rate of the pump 15 which is stored in the flash memory 63, as shown at step S33. At the following step S34, the control section 21 determines whether information on the liquid supply rate of the pump has been retrieved from the flash memory 63, in other words, whether information on the liquid supply rate is stored in the flash memory 63. If it cannot obtain information on the liquid supply rate, the control section 21 returns to step S33 after performing processing at step S35. If it was able to obtain liquid supply rate information, the control section 21 proceeds to step S36.

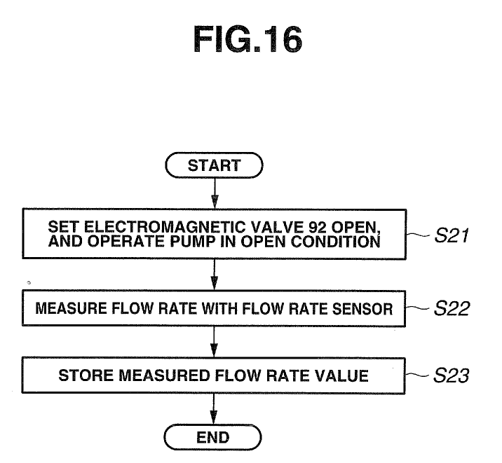

At step S35, the control section 21 performs operation control for processing for measuring the liquid supply rate of the pump 15 and storing the rate in the flash memory 63 as shown in FIG. 16.

As shown at step S21 of FIG. 16, the control section 21 runs the pump 15 with the electromagnetic valve 92 open, and the electromagnetic valve 91 and the electromagnetic valves 18a to 18c, which are connected to load, closed. That is to say, the pump 15 is run in a released condition (or a condition near a no load condition). In this case, the control section 21 further switches the change-over valve 29 so that the valve 29 communicates with the electromagnetic valve 92, and discharges washing water that has flown through the change-over valve 29 to outside through liquid supplying operation of the pump 30. Washing water may also be brought back into the washing and disinfecting bath 3 instead of being discharged to the outside.

At the following step S22, the flow rate sensor 17 measures a flow rate in a case where liquid is supplied by the pump 15 in a released condition. Then, at the following step S23, the control section 21 obtains a measured flow rate value from the flow rate sensor 17 and stores the value in the flash memory 63. In this way, processing for measuring and storing the liquid supply rate of the pump 15 shown in FIG. 16 is terminated. Then, the control section 21 returns to processing at step S33 in FIG. 15 and proceeds to processing at step S36 from step S34.

At step S36, the control section 21 recognizes a proper flow rate range in a case where liquid is supplied to channels from information in channels of the endoscope 2 and information on the liquid supply rate of the pump 15. In other words, the control section 21 sets a threshold value for determining whether there is clogging or not when liquid is supplied to each channel.

At the following step S37, the control section 21 controls various sections of the main body 5 to supply washing water into suction channels, gas/water supply channels, and a wire insertion channel in sequence to start a washing process.

In this case, as shown at step S38, the control section 21 periodically has the flow rate sensor 17 measure flow rate and obtains a measured flow rate. As processing after step S38, processing at step S9 and subsequent steps of FIG. 4 is performed.

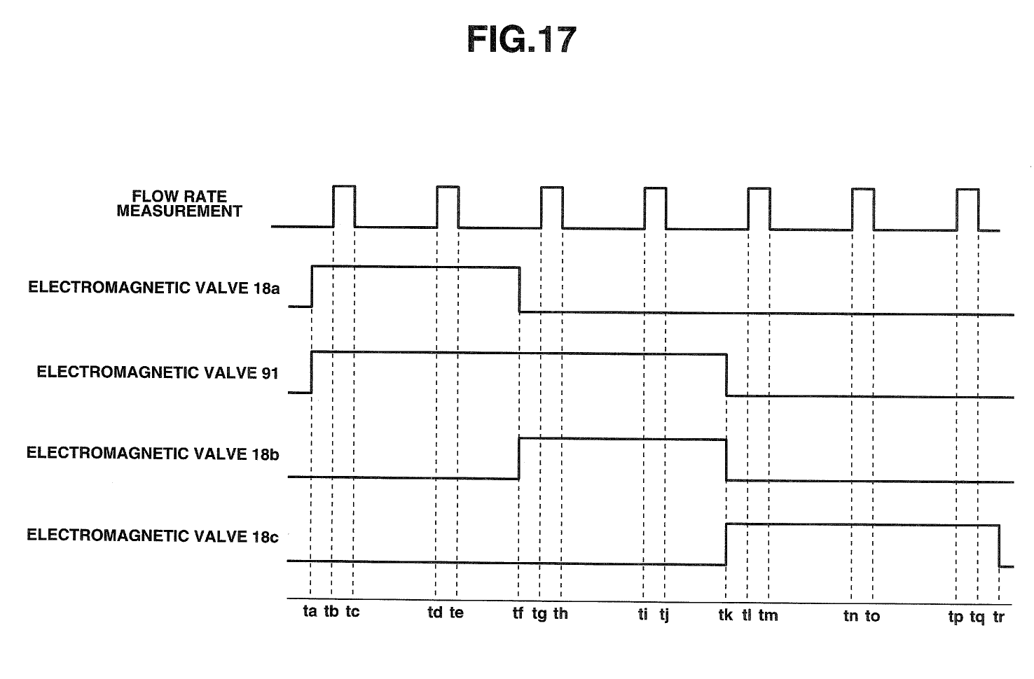

FIG. 17 is a diagram that illustrates flow rate measuring operations in the washing process at steps S37 and S38. FIG. 17 shows opening/closing control of the electromagnetic valves 18a to 18c and 91 with the electromagnetic valves 18d and 18e eliminated from FIG. 6 and the electromagnetic valve 91 added. As the electromagnetic valve 92 is closed all the time, it is not shown in FIG. 17.

As shown in FIG. 17, the electromagnetic valve 18a is opened during time of washing a suction channel, ta-tf, and the electromagnetic valve 91 is closed during this time, ta-tf. And the control section 21 performs washing while monitoring whether a flow rate in the suction channel is within a proper range based on the flow rate that flows to the side of the flow rate sensor 17.

In this case, flow rate control with accurate flow rate determination is possible because the control section 21 determines whether the flow rate to the flow rate sensor 17 is within a proper range or not in the case of the suction channel from the value of the liquid supply rate of the pump 15.

Also, as shown in FIG. 17, during time of washing gas/water supply channels, tf-tk, the electromagnetic valve 18b is opened, and the electromagnetic valve 91 is opened during this time, tf-tk. Then, the control section 21 performs washing while monitoring whether a flow rate on the gas/water supply channels is within a proper range based on the flow rate that flows to the side of the flow rate sensor 17.

In this case, flow rate control with accurate flow rate determination is possible because the control section 21 determines whether the flow rate to the flow rate sensor 17 is within a proper range or not in the case of the gas/water supply channels based on the value of the liquid supply rate of the pump 15.

Meanwhile, as shown in FIG. 17, during time of washing a wire insertion channel, tk-tr, the electromagnetic valve 18e is opened, and the electromagnetic valve 91 is closed during this time, tk-tr. And the control section 21 performs washing while monitoring whether a flow rate to the flow rate sensor 17 side, that is, the flow rate that flows to the wire insertion channel, is within a proper range.

Since the control section 21 determines whether the flow rate is within a proper range or not in the case of the wire insertion channel based on the value of the liquid supply rate of the pump 15 also in this case, flow rate control with accurate flow rate determination is possible. While FIG. 17 describes operations of the washing process, almost the same flow rate control is performed in other processes as well.

According to the present embodiment, by diverting part of flow to the flow rate sensor 17 through the bypass channel 14h, it is possible to perform flow rate control with accurate detection of flow rate with only one flow rate sensor 17 without requiring the orifice 19a which constitutes a flow rate limiting section. Specifically, when the suction channel or the like of the endoscope 2 is washed or disinfected at a flow rate exceeding the flow rate measurement range of the flow rate sensor 17, the electromagnetic valve 91 in the bypass channel 14h which is provided in parallel with the flow rate sensor 17 is opened to divert part of the flow. By diverting part of the flow, flow rate can be accurately measured within the measurement range of the flow rate sensor 17.

In addition, since the present embodiment measures and stores the liquid supply rate of the pump 15, which constitutes a fluid supply unit, and determines whether flow rate is proper or not by using the measured liquid supply rate, it can accurately detect flow rate and also accurately detect any condition with deviation from a proper flow rate. To be specific, it is possible to detect a condition in which a channel is completely clogged as well as a condition in which soil, for example, at the time of an internal examination adheres to an inner side of a channel to reduce flow rate in the channel.

Additionally, because the present embodiment adopts a configuration that does not require limitation on flow rate even in a channel of a large inner diameter, such as a suction channel (in this case, flow that flows to the flow rate sensor 17 portion is limited to within the measurement range), washing and/or disinfection can be performed with a flow rate appropriate for the inner diameter of a channel even when various types of channels are of different inner diameters.

Therefore, washing and/or disinfection can be completed in a smaller amount of time than when flow rate is limited.

As a variation of the present embodiment, a plurality of the bypass channels 14h and electromagnetic valves 91 which are arranged in parallel with the flow rate sensor 17 may be provided, so that flow rate or the like can be accurately detected even for channels of a wider variety of inner diameters by controlling opening/closing of two electromagnetic valves 91.

Additionally, an embodiment or the like that is formed such as by combining part of the above-described embodiments or the like also belongs to the present invention.

For example, the present invention is not limited to a configuration that includes only one flow rate limiting section, one flow rate padding section, and one flow rate diverting section of the above-described embodiments but may be applied to a configuration that includes a plurality of such sections. In a configuration including a plurality of such sections, any section that can measure flow rate more accurately can be selected for use.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

1. An endoscope washing and disinfecting apparatus, comprising:
a fluid supply unit that supplies fluid for washing and disinfecting;
a plurality of connecting channels which are connected to a plurality of channels of an endoscope;
an electromagnetic valve provided in each of the plurality of connecting channels;
a single flow rate meter, provided between the fluid supply unit and the electromagnetic valve; and
a flow rate limiting section for limiting flow rate to at least a channel in which the fluid flows at a flow rate exceeding a flow rate measurement range in which flow rate measurement by the flow rate meter is possible among the plurality of channels so that the flow rate falls within the flow rate measurement range; or
a flow rate padding section for padding a flow rate measured by the flow rate meter with a flow rate that can be detected by the flow rate meter at least for a channel in which the fluid flows at a flow rate that does not reach a lower limit value of the flow rate measurement range among the plurality of channels; or
a flow rate diverting section for diverting part of flow that flows to the flow rate meter through a bypass channel which is opened and closed in parallel with the flow rate meter so that flow rate falls within the flow rate measurement range of the flow rate meter.

2. The endoscope washing and disinfecting apparatus according to claim 1, further comprising
a determination section for determining whether or not a flow rate measured by the flow rate meter in a case where the fluid is flown in each of the plurality of channels deviates from a predetermined range which is set for each of the plurality of channels.

3. The endoscope washing and disinfecting apparatus according to claim 1, wherein
in a case where the flow rate limiting section is provided, the flow rate limiting section comprises an orifice for limiting fluid that is provided in a connecting channel connected to at least a channel of a largest inner diameter among the plurality of channels of the endoscope.

4. The endoscope washing and disinfecting apparatus according to claim 1, wherein
in a case where the flow rate padding section is provided, when a flow rate at least in a channel of a smallest inner diameter among the plurality of channels of the endoscope is measured, the flow rate padding section pads a flow rate measured by the flow rate meter with a flow rate that flows to other channel different from the channel of the smallest inner diameter while opening the electromagnetic valve provided in the connecting channel connected to the other channel.

5. The endoscope washing and disinfecting apparatus according to claim 1, wherein
in a case where the flow rate diverting section is provided, the flow rate diverting section opens an electromagnetic valve inserted in the bypass channel when measuring a flow rate of fluid flowing in at least a channel of the largest inner diameter among the plurality of channels of the endoscope.

6. The endoscope washing and disinfecting apparatus according to claim 1, wherein
in a case where the flow rate limiting section is provided, a bypass connecting channel which is opened and closed is provided in parallel with the flow rate limiting section, and selection between passing through the flow rate limiting section and passing through the bypass connecting channel is enabled.

7. (canceled)

7. The endoscope washing and disinfecting apparatus according to claim 1, further comprising
an identification information reading section for reading identification information specific to the endoscope in a non-contact manner.

8. The endoscope washing and disinfecting apparatus according to claim 2, further comprising
an identification information reading section for reading identification information specific to the endoscope in a non-contact manner.

9. The endoscope washing and disinfecting apparatus according to claim 1, wherein
the fluid supply unit comprises a pump for supplying the fluid to the plurality of channels of the endoscope, and the endoscope washing and disinfecting apparatus comprises a flow rate measuring section for measuring a flow rate of liquid supplied by the pump with the flow rate meter when the pump is being switched to a released condition in which the fluid is not supplied to the plurality of channels of the endoscope.

10. The endoscope washing and disinfecting apparatus according to claim 1, further comprising
a pump for supplying the fluid to the plurality of channels of the endoscope, and a flow rate measuring section for measuring a flow rate of liquid supplied by the pump with the flow rate meter when the pump is being switched to a released condition in which the fluid is not supplied to the plurality of channels of the endoscope.

11. The endoscope washing and disinfecting apparatus according to claim 10, further comprising:
a storing section for storing information on a flow rate measured by the flow rate measuring section; and
a determination section for determining whether or not a flow rate in each of the plurality of channels which is measured by the flow rate meter is within a predetermined range using the information.

12. The endoscope washing and disinfecting apparatus according to claim 1, further comprising
a control section for controlling opening and closing of the electromagnetic valve.

13. The endoscope washing and disinfecting apparatus according to claim 7, further comprising
a control section for controlling opening and closing of the electromagnetic valve using identification information read by the identification information reading section.

14. The endoscope washing and disinfecting apparatus according to claim 6, wherein
the bypass connecting channel is opened during time in which flow rate measurement with the flow rate meter is not performed.

15. The endoscope washing and disinfecting apparatus according to claim 4, wherein
the flow rate padding section further calculates a flow rate in the channel of the smallest inner diameter by subtracting a flow rate in the other channel from a flow rate of the fluid meter which is measured being padded with the flow rate in the other channel.

16. The endoscope washing and disinfecting apparatus according to claim 1, further comprising
a channel information storing section in which channel information including inner diameters of a plurality of channels provided in multiple types of endoscopes is stored.

17. An endoscope washing and disinfecting method for washing and disinfecting a plurality of channels of an endoscope with fluid supplied from a fluid supply unit, the method comprising:
a flow rate monitoring step of monitoring a flow rate in each of the plurality of channels by means of a single flow rate meter provided between the fluid supply unit and an electromagnetic valve provided in each of a plurality of connecting channels connected to each of the plurality of channels, wherein
the flow rate monitoring step employs:
a flow rate limiting step of limiting flow rate to at least a channel in which the fluid flows at a flow rate exceeding a flow rate measurement range in which flow rate measurement by the flow rate meter is possible among the plurality of channels so that the flow rate falls within the flow rate measurement range; or
a flow rate padding step of padding a flow rate measured by the flow rate meter with a flow rate that can be detected by the flow rate meter at least for a channel in which the fluid flows at a flow rate that does not reach a lower limit value of the flow rate measurement range among the plurality of channels; or
a flow rate diverting step of diverting part of flow rate that flows to the flow rate meter through a bypass channel which is opened and closed in parallel with the flow rate meter so that flow rate falls within the flow rate measurement range of the flow rate meter.

18. The endoscope washing and disinfecting method according to claim 17, further comprising
a determination step of determining whether a flow rate monitored by the flow rate meter within the flow rate measurement range deviates from a predetermined range or not.

19. The endoscope washing and disinfecting method according to claim 17, further comprising
a channel information obtaining step of obtaining channel information including inner diameter of the plurality of channels of the endoscope which are removably connected to the plurality of connecting channels.

20. The endoscope washing and disinfecting method according to claim 17, wherein
the flow rate monitoring step is periodically performed in a time period in which fluid flows in each of the plurality of channels.

21. The endoscope washing and disinfecting apparatus according to claim 1, further comprising:
a pressure gauge for measuring pressure of the channel of the smallest inner diameter, in the connecting channel which is connected to at least the channel of the smallest inner diameter among the plurality of channels of the endoscope.
